(19) United States
(12) Patent Application Publication
(10) Pub. No.: US 2013/0006067 A1
(43) Pub. Date: Jan. 3, 2013

(54) METHOD AND DEVICE FOR DETERMINING A RISK OF GRAFT REJECTION

(76) Inventors: Yohann Foucher, Nantes (FR); Magali Giral, Carquefou (FR); Jean-Paul Soulillou, Nantes (FR); Pascal Daguin, Nantes (FR)

(21) Appl. No.: 13/516,707

(22) PCT Filed: Dec. 15, 2010
(86) PCT No.: PCT/EP2010/069785

§ 371 Date: Sep. 17, 2012

(30) Foreign Application Priority Data
Dec. 16, 2009 (FR) 0959043

(51) Int. Cl. A61B 5/145

(52) U.S. Cl. 600/309

(57) ABSTRACT

The invention relates to a method for determining a risk of permanent rejection of a graft after being transplanted to a recipient, said method being characterized in that it includes the following steps: (a) entering physiological/clinical characteristics of the recipient and the donor into a computation unit by means of an input interface, said characteristics including the gender of the recipient, the creatinine level of the graft donor when sampled, the age of the recipient at the time of the transplant,—the number of renal transplants previously received by the recipient,—a creatinine level of the recipient measured twice at at least one predetermined point in time after the transplant, a proteinuria level of the recipient measured at a predetermined point in time after the transplant, and the existence of an episode of permanent rejection of the transplant during the first year of the transplant; (b) determining the parameters on the basis of said characteristics; (c) combining said parameters so as to obtain a risk score of permanent rejection of the gait; and (d) analyzing said risk score so as to determine a risk of permanent rejection of a graft.

The invention relates to the field of organ transplantations (or graft) into a patient, specifically the field of kidney transplantations.

More particularly, the invention relates to the care of patients having undergone kidney transplantation.

More particularly still, the invention relates to estimating (or determining) the risk of permanent rejection of an organ, notably a kidney.

The expression “permanent rejection” refers to rejection of the transplant requiring a permanent return to dialysis.

In the past, conventional evaluation criteria for estimating the effectiveness of new agents were measured in the short term. This is the case, for example, for cyclosporine (or cisclosporin), which is an immunosuppressor whose therapeutic use has enabled major improvements in organ survival (and the long-term survival of patients).

The significant increase in survival rate of a transplanted organ associated with a course of cyclosporine led to consideration of rejection during the first year following transplantation as an evaluation criterion for the effectiveness of new products such as anti-IL2 receptor antibodies, mycophenolate mofetil and tacrolimus.

However, the significant decrease in the permanent rejection rate over the past few years has not been associated with the development of substitute evaluation criteria concerning the risk of transplant rejection, notably in the long term.

It has been proposed to turn to analyses of biopsies in order to obtain an evaluation criterion for the long-term risk of transplant rejection. Although such a procedure has certain determination effectiveness, it remains an invasive, dangerous and costly method.

More recently still, noninvasive biomarkers (collected from urine or blood) have been proposed for determining risk of rejection such as perforin, granzyme B, CD30 and antibodies of specific donors.

However, such biomarkers require subsequent validations on large populations of patients, and often appear too late to be substitute markers (sometimes after a period of 15 to 20 months).

Creatinine estimated at six and/or 12 months after the transplantation is regarded as a marker with a good correlation with transplant survival. However, in spite of this correlation, criticism has been raised, denouncing creatinine as a marker with little predictive utility.

Similarly, kidney function, namely the assay of creatinine clearance in the blood, is commonly used in clinical practice and in therapeutic tests as an evaluation criterion, although much criticism has been raised in terms of this criterion's reliability.

Consequently, there is currently no reliable tool for determining in an early and noninvasive manner the long-term risk of permanent rejection of a kidney transplant.

One goal of the present invention is to propose an early method (in the first year after the transplantation) and an associated device for determining the long-term risk of permanent rejection of a transplant.

Another goal of the present invention is to offer a method for evaluating a risk of permanent rejection, this method being simple to implement and noninvasive.

Another goal of the present invention is to offer a method that is more reliable than methods for determining the risk of permanent rejection proposed by the state of the art.

To that end, the invention relates to a method for determining a risk of permanent rejection of an organ after being transplanted to a recipient, characterized in that it includes the following steps:
(a) entering physiological/clinical characteristics of the recipient and the donor using an input interface into a computation unit, said characteristics including:
   the recipient's gender,
   the transplant donor's creatinine level when sampled,
   the recipient's age at the time of the transplantation,
   the number of kidney transplants previously received by the recipient,
   a creatinine level of the recipient measured at two at least one predetermined point in time after the transplantation,
   a recipient's proteinuria level measured at a predetermined point in time after the transplantation, and
   the existence of an episode of acute rejection during the first year of transplantation,
(b) determining the parameters on the basis of said characteristics,
(c) combining said parameters so as to obtain a permanent transplant rejection risk score, and
(d) analyzing said risk score so as to determine a permanent transplant rejection risk.

The expression “transplant donor's creatinine level when sampled” refers to the donor's creatinine level before removal of the organ for said transplantation.

Such a method provides after one year following the transplantation procedure a score associated with permanent rejection of the transplanted kidney in the long term, typically eight years following the transplantation.

The term “recipient” obviously refers to the patient who receives the transplant during a transplantation and “donor” refers to the person from which said organ is removed.

Advantageously, but optionally, the invention includes at least one of the following characteristics:
the characteristics include two creatinine levels of the recipient measured at two predetermined points in time after the transplantation,
the two predetermined points in time are three months and 12 months after the transplantation,
proteinuria level is measured 12 months after the transplantation,
the parameter determination step includes the determination of a Boolean parameter by comparing the donor's creatinine level when sampled with a threshold value,
the parameter determination step includes the determination of a Boolean parameter by comparing the recipient's age and a threshold value,
the parameter determination step includes the determination of a Boolean parameter by comparing the number of kidney transplants previously received by the recipient with a threshold value,
the parameter determination step includes the determination of a numerical parameter for each creatinine level expressed in a predetermined unit,
the parameter determination step includes the determination of at least one numerical parameter of proteinuria level expressed in a predetermined unit,
the parameter determination step includes the determination of a Boolean parameter as a function of the existence or absence of an episode of acute rejection reported in the first year of transplantation,
the combination is a weighted total,
said combination is obtained by implementation in the computation unit of the following formula:

KTFS=K₁*Cr_{—}D+K₂*Age_{—}R+K₃*Ntrans+K₄*AR+K₅*Cr_{—}3m+K₆*√(Cr_{—}12m)+K₇*Gender+K₈*Pr_{—}12m+K₉*(Pr_{—}12m)²+K₁₀*Gender*Pr_{—}12m+K₁₁*Gender*(Pr_{—}12m)²
wherein:
   KTFS is the risk score,
   Cr_D is equal to 1 if the donor's creatinine level when sampled is greater than a threshold value and 0 if otherwise,
   Age_R is equal to 1 if the recipient's age is greater than a threshold value and 0 if otherwise,
   Ntrans is equal to 1 if the number of kidney transplants previously received by the recipient is greater than a threshold value and 0 if otherwise,
   AR is equal to 1 if at least one episode of acute rejection was reported by the recipient in the first year of transplantation and 0 if otherwise,
   Cr_{—}3m is the recipient's creatinine level measured at a first predetermined point in time after the transplantation,
   Cr_{—}12m is the recipient's creatinine level measured at a second predetermined point in time after the transplantation,
   Pr_{—}12m is the recipient's proteinuria level measured at a predetermined point in time after the transplantation,
   Gender is equal to 1 if the recipient is male and 0 if the recipient is female, and
   K₁ to K₁₁ are coefficients.
the threshold value for the donor's creatinine level is 190 μmol/l, creatinine levels of the recipient are expressed in μmol/l, the first and second predetermined points in time are three months and 12 months, respectively, proteinuria level is expressed in g/day, the predetermined point in time for proteinuria level is 12 months,
the parameters K₁ to K₁₁ are selected from the following ranges:
K₁ is selected in a range from −1.04 to −0.41,
K₂ is selected in a range from −1.10 to −0.92,
K₃ is selected in a range from 0.89 to 1.26,
K₄ is selected in a range from 0.20 to 0.33,
K₅ is selected in a range from −0.03 to 0.03,
K₆ is selected in a range from 0.34 to 0.48,
K₇ is selected in a range from −0.94 to −0.76,
K₈ is selected in a range from 0.50 to 0.69,
K₉ is selected in a range from −0.08 to 0.13,
K₁₀ is selected in a range from 0.38 to 0.60,
K₁₁ is selected in a range from −0.25 to 0.15,
the parameters K₁ to K₁₁ have the following values:
K₁=−0.7265,
K₂=−1.0102,
K₃=1.0748,
K₄=0.2639,
K₅=0.0011,
K₆=0.4108,
K₇=−0.8529,
K₈=0.5925,
K₉=0.0230,
K₁₀=0.4876,
K₁₁=−0.0508,
analysis of the risk score includes a step of comparing said score with a threshold value, a score greater than the value threshold meaning than the recipient is considered as at risk of returning to dialysis,
the threshold value is 4.04.

The invention also relates to a product/computer program containing a set of instructions characteristic of the implementation of the inventive method.

The invention also relates to a processing system including a computation unit and an input interface, characterized in that said system includes means for implementing the inventive method.

Other characteristics, aims and advantages of the present invention will become apparent upon consideration of the detailed description which follows, with respect to the appended figures given as nonrestrictive examples wherein:

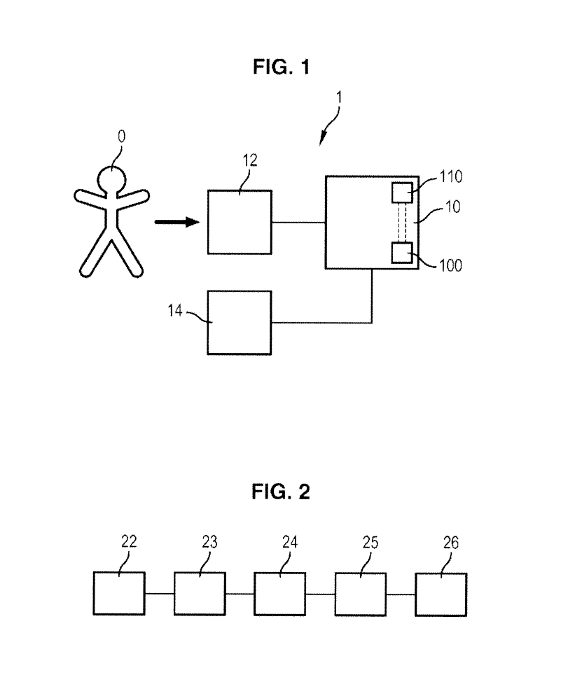

FIG. 1 is a schematic representation of a processing system according to a particular embodiment of the present invention,

FIG. 2 is a functional graph representing a method according to a particular embodiment of the present invention,

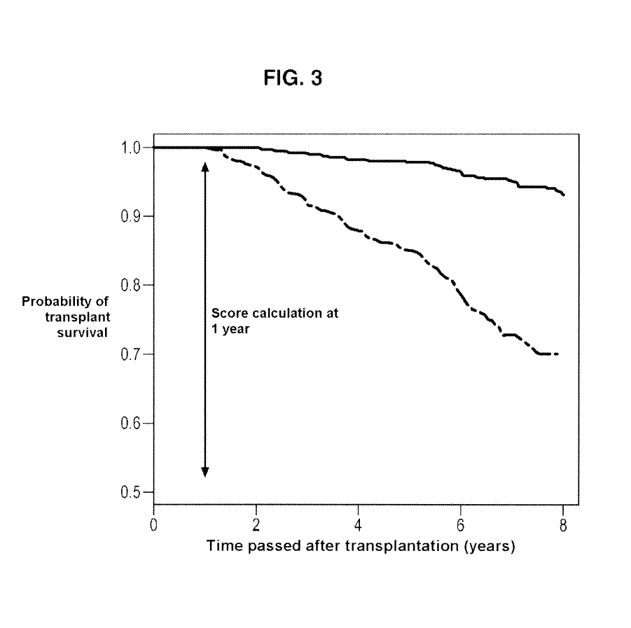

FIG. 3 is a survival probability graph for a transplanted organ.

In reference to FIG. 1, a device (1) according to a particular embodiment of the present invention includes a computation unit (10) capable of following computer instructions and processing data. One such computation unit preferentially includes a microprocessor (110) which can be of any type known in the state of the art. The computation unit (10) also has a storage unit (100) that is capable of receiving a computer program including a set of instructions characteristic of the implementation of the method, and is capable of storing data.

The device (1) also includes an input interface (12) connected to the computation unit (10) enabling an operator (O) of the device (1) to enter data to be treated. One such input interface (12) includes any element enabling the entry of such data destined for the computation unit (10) such as a keyboard element optionally associated with a pointing device element.

Preferentially, the computation unit further includes an output interface (14) such as a screen that on the one hand enables the user to verify the integrity of the data entered but on the other hand enables the computation unit (10) to be able to interact with the operator (O).

The device (1) can be integrated in a single system such as a computer, a smartphone or any other system known in the state of the art enabling implementation of the inventive method. The operator (O) can be of any skill level and thus may or may not have medical qualifications.

It is notably envisaged according to a particular embodiment of the present invention that the data entered by the operator (O) are sent via a network (the Internet, for example) preferentially in a secure manner to a remote server comprising a computation unit capable of implementing the inventive method and thus of treating the data received by the server. Optionally, after said processing, the server returns the result of the analysis to the user via the same network or another. Optionally, the server records the data and/or the result of the analysis on a means of recording. Obviously, means of guaranteeing the anonymity of the physiological/clinical characteristics of the donor and the recipient can be envisaged.

Thus, one such device (1) enables implementation of the inventive method, i.e., it enables implementation of the following steps:
entering physiological/clinical characteristics using the input interface (12) into a computation unit (10) (step 22), said characteristics including:
   the recipient's gender,
   the transplant donor's creatinine level when sampled,
   the recipient's age at the time of the transplantation,
   the number of kidney transplants previously received by the recipient,
   the recipient's creatinine level measured at at least two predetermined points in time after the transplantation,
   the recipient's proteinuria level measured at a predetermined point in time after the transplantation,
   the existence of an episode of acute rejection in the first year following transplantation,
determining the parameters on the basis of said characteristics via data processing by the computation unit (10) (step 23),
combining said parameters so as to obtain a transplant rejection risk score via data processing by the computation unit (10) (step 24), and
analyzing said risk score so as to determine a permanent transplant rejection risk (step 25).

Thus, the calculated score makes it possible to determine the risk of long-term transplant rejection by a recipient after kidney transplantation. It is based on a synthesis of a plurality of clinical risk factors.

One such method has a dual advantage:
it constitutes a tool for assisting medical decision-making by providing an early determination of the risk of return to dialysis without any invasive intervention and based on easily measurable and thus inexpensive clinical parameters,
it makes it possible to evaluate the long-term progress of a transplant patient and can thus also be used as a judgment criterion in clinical studies which could thus be shortened.

Thus, the inventive method can be implemented not only by clinical or hospital personnel but also by all persons involved in clinical research (pharmaceutical industry, scientists, doctors, etc.).

The score resulting from the inventive method is more precisely based on the weighted total of the clinical characteristics.

The weights were determined statistically by use of the linear predictor of the Cox model corrected by maximization of the area under the ROC curve dependent on time at 8 years after the transplantation. The time studied is the period between transplantation and return to dialysis. Three-thousand forty patients from France's DIVAT (“Données Informatisées et Validées en Transplantation” or Digitized and Validated Transplantation Data) observational cohort were included for the estimation of this model.

The model advantageously takes into account the characteristics including two creatinine levels of the recipient measured at two predetermined points in time after the transplantation. Indeed, it was noted that the consideration of these characteristics was relevant. More precisely, the two predetermined points in time are roughly three months and 12 months after the transplantation.

Moreover, this model established the relevance of proteinuria measurements 12 months after the transplantation. Indeed, the consideration of such a characteristic was relevant.

An important point of the model is the determination of parameters related to the characteristics. Notably, several ways of determining the parameters are envisaged:
determination of a Boolean parameter by comparing the donor's creatinine level when sampled with a threshold value,
determination of a Boolean parameter by comparing the recipient's age at the time of the transplantation with a threshold value,
determination of a Boolean parameter by comparing the number of kidney transplants previously received by the recipient with a threshold value,
determination of a numerical parameter for each creatinine level expressed in a predetermined unit,
determination of at least one numerical parameter of proteinuria level expressed in a predetermined unit,
determination of a Boolean parameter as a function of the existence or absence of an episode of acute rejection reported in the first year following transplantation.

The specific model is advantageously a combination of these parameters and more precisely a weighted total.

According to a preferred embodiment, the score is based on a formula of the following type:

KTFS=K₁*Cr_{—}D+K₂*Age_{—}R+K₃*Ntrans+K₄*AR+K₅*Cr_{—}3m+K₆*√(Cr_{—}12m)+K₇*Gender+K₈*Pr_{—}12m+K₉*(Pr_{—}12m)²+K₁₀*Gender*Pr_{—}12m+K₁₁*Gender*(Pr_{—}12m)²

wherein:
KTFS is the permanent rejection risk score calculated in an early manner,
Cr_D is equal to 1 if the donor's creatinine level at the time of the transplantation is greater than a threshold value and 0 if otherwise,
Age_R is equal to 1 if the recipient's age is greater than a threshold value and 0 if otherwise,
Ntrans is equal to 1 if the number of kidney transplants previously received by the recipient is greater than a threshold value and 0 if otherwise,
AR is equal to 1 if at least one episode of permanent rejection was reported in the first year following a previous transplantation and 0 if otherwise,
Cr_{—}3m and Cr_{—}12m are the recipient's creatinine levels measured at at least two predetermined points in time after the transplantation,
Pr_{—}12m is the recipient's proteinuria level measured at a predetermined point in time after the transplantation,
Gender is equal to 1 if the recipient is male and 0 if the recipient is female, and
K₁ to K₁₁ are weighting coefficients.

The following table summarizes the clinical characteristics and the value of their associated parameter:

| Abbreviation | Value |
|---|---|
| Gender | 1 if the recipient is male and 0 if |
| | female |
| Cr_D | 1 if the donor's blood creatinine is |
| | greater than 190 μmol/l and 0 if not |
| Age_R | 1 if the recipient's age at the time of |
| | the transplantation is greater than 25 |
| | years and 0 if not |
| Ntrans | 1 if the number of kidney transplants |
| | previously received by the recipient is |
| | greater than 2 or 0 if not |
| Cr_3m | The recipient's blood creatinine in |
| | μmol/l measured three months after the |
| | transplantation |
| Cr_12m | The recipient's blood creatinine in |
| | μmol/l measured one year after the |
| | transplantation |
| Pr_12m | The recipient's proteinuria in g/day |
| | measured one year after the |
| | transplantation |
| AR | 1 if an episode of permanent rejection |
| | was reported for the recipient in the |
| | first year of a previous transplantation |
| | and 0 if not |

The parameters K₁ to K₁₁ are advantageously selected from the following ranges:

K₁ is selected in a range from −1.04 to −0.41,

K₂ is selected in a range from −1.10 to −0.92,

K₃ is selected in a range from 0.89 to 1.26,

K₄ is selected in a range from 0.20 to 0.33,

K₅ is selected in a range from −0.03 to 0.03,

K₆ is selected in a range from 0.34 to 0.48,

K₇ is selected in a range from −0.94 to −0.76,

K₈ is selected in a range from 0.50 to 0.69,

K₉ is selected in a range from −0.08 to 0.13,

K₁₀ is selected in a range from 0.38 to 0.60,

K₁₁ is selected in a range from −0.25 to 0.15.

Preferentially, the parameters K₁ to K₁₁ have the following values:

K₁=−0.7265,

K₂=−1.0102,

K₃=1.0748,

K₄=0.2639,

K₅=0.0011,

K₈=0.4108,

K₇=−0.8529,

K₈=0.5925,

K₉=0.0230,

K₁₀=0.4876,

K₁₁=−0.0508.

The result is the following formula:

KTFS=−0.72649*Cr_{—}D−1.01017*Age_{—}R+1.07482*Ntrans+0.26395*AR+0.00114*Cr_{—}3m+0.41084*√(Cr_{—}12m)−0.85292*Gender+0.59253*Pr_{—}12m+0.02298*(Pr_{—}12m)²+0.48762*Gender*Pr_{—}12m−0.05078*Gender*(Pr_{—}12m)²

The higher the final score, the greater the risk of returning to dialysis.

It should be noted, however, that this score was determined specifically for kidney transplantations and is preferentially applicable for recipients over 18 years of age.

The inventive method makes it possible to study simultaneously information provided by several clinical characteristics that are irrelevant or unreliable when considered in isolation.

Indeed, the determination and combination of parameters resulting from said characteristic make it possible to offer a method for determining the permanent transplant rejection risk that is more reliable than methods of the state of the art. The use of parameters makes it possible to construct a predictive score without inevitably seeking to directly interpret the source characteristics as is the case in the evaluation methods of the state of the art. It is notably this severing of the clinical characteristics from the interpretation thereof via the development of parameters that enables improved determination of the risk of permanent rejection signifying a return to dialysis.

Consequently, one such method helps improve patient care by monitoring suited to the patient's risk of permanent rejection and it enables optimization of resources toward the most fragile patients.

Moreover, the inventive method makes it possible to shorten certain studies, in particular industrial protocols, and thus makes it possible to reduce costs and to improve the benefit/risk balance to the advantage of patients.

The score resulting from the inventive method was validated for a prediction up to eight years after the transplantation.

The totality of the study undertaken made it possible to establish a threshold for making a binary prognostic decision (return to dialysis or functional transplant): above 4.04, patients are considered at risk of returning to dialysis in the first eight years following transplantation. Obviously, any other threshold can be established for a binary prognosis according to predictive needs and constraints.

In reference to FIG. 3, which illustrates kidney transplant survival in the first eight years after the transplantation as a function of the score calculated one year after the transplantation, two groups of patients can be clearly identified as a function of the risk of returning to dialysis. In this figure, the x-axis represents time passed (in years) since transplantation and the y-axis the probability of transplant survival expressed on a scale from 0 to 1.

In the first group (solid line) corresponding to a score below or equal to 4.04, the probability of returning to dialysis before eight years post-transplant is roughly 7%. This probability is on the order of 30% (high risk) in the second group (broken line) corresponding to a score greater than 4.04. The inventive method was tested and validated on an external sample of 343 patients.

The inventive method is only suited for kidney transplantation.

The inventive method can advantageously be used as a determination criterion, whether primary or secondary, in research studies or protocols. Indeed, using this method, it is possible to measure in an early manner a permanent transplant rejection risk and to enable studies that are shorter and thus less risky for patients and cheaper for study sponsors.

The reliability of the permanent rejection risk determination of the inventive method exceeds that of creatinine or proteinuria, even though these two markers are commonly selected as evaluation criteria.

The inventive method also advantageously includes a preliminary step of measuring the donor's creatinine when sampled.

It is quite evident that in order to further improve the determination of the risk of permanent rejection, one or more other characteristics can be taken into account and can be thus introduced during step (a).

1. A method for determining a risk of permanent rejection of an organ after being transplanted to a recipient, characterized in that said method includes the following steps:
(a) entering physiological/clinical characteristics of the recipient and the donor using an input interface into a computation unit, said characteristics including:
the recipient's gender,
the transplant donor's creatinine level when sampled,
the recipient's age at the time of the transplantation,
the number of kidney transplants previously received by the recipient,
a recipient's creatinine level measured at two at least one predetermined point in time after the transplantation,
a recipient's proteinuria level measured at a predetermined point in time after the transplantation, and
the existence of an episode of acute rejection during the first year of transplantation,
(b) determining the parameters on the basis of said characteristics,
(c) combining said parameters so as to obtain a permanent transplant rejection risk score, and
(d) analyzing said risk score so as to determine a permanent transplant rejection risk.

2. The method of claim 1, wherein the characteristics include two creatinine levels of the recipient measured at two predetermined points in time after the transplantation.

3. The method of claim 2, wherein the two predetermined points in time are 3 months and 12 months after the transplantation.

4. The method of claim 1, wherein the proteinuria level is measured 12 months after the transplantation.

5. The method of claim 1, wherein the parameter determination step includes the determination of a Boolean parameter by comparing the donor's creatinine level when sampled with a threshold value.

6. The method of claim 1, wherein the parameter determination step includes the determination of a Boolean parameter by comparing the recipient's age and a threshold value.

7. The method of claim 1, wherein the parameter determination step includes the determination of a Boolean parameter by comparing the number of kidney transplants previously received by the recipient with a threshold value.

8. The method of claim 1, wherein the parameter determination step includes the determination of a numerical parameter for each creatinine level expressed in a predetermined unit.

9. The method of claim 1, wherein the parameter determination step includes the determination of at least one numerical parameter of proteinuria level expressed in a predetermined unit.

10. The method of claim 1, wherein the parameter determination step includes the determination of a Boolean parameter as a function of the existence or absence of an episode of acute rejection reported in the first year of transplantation.

11. The method of claim 1, wherein said combination is a weighted total.

12. The method of claim 11, wherein said combination is obtained by implementation in the computation unit of the following formula:
KTFS=K₁*Cr_{—}D+K₂*Age_{—}R+K₃*Ntrans+K₄*AR+K₅*Cr_{—}3m+K₆*√(Cr_{—}12m)+K₇*Gender+K₈*Pr_{—}12m+K₉*(Pr_{—}12m)²+K₁₀*Gender*Pr_{—}12m+K₁₁*Gender*(Pr_{—}12m)²
wherein:
KTFS is the risk score,
Cr_D is equal to 1 if the donor's creatinine level when sampled is greater than a threshold value and 0 if otherwise,
Age_R is equal to 1 if the recipient's age is greater than a threshold value and 0 if otherwise,
Ntrans is equal to 1 if the number of kidney transplants previously received by the recipient is greater than a threshold value and 0 if otherwise,
AR is equal to 1 if at least one episode of acute rejection by the recipient was reported in the first year of transplantation and 0 if otherwise,
Cr_{—}3m is the recipient's creatinine level measured at a first predetermined point in time after the transplantation,
Cr_{—}12m is the recipient's creatinine level measured at a second predetermined point in time after the transplantation,
Pr_{—}12m is the recipient's proteinuria level measured at a predetermined point in time after the transplantation,
Gender is equal to 1 if the recipient is male and 0 if the recipient is female, and
K₁ to K₁₁ are coefficients.

13. The method of claim 12, wherein the threshold value for the donor's creatinine level is 190 μmol/l, the recipient's creatinine levels are expressed in μmol/l, the first and second predetermined points in time are three months and 12 months, respectively, proteinuria level is expressed in g/day, the predetermined point in time for proteinuria level is 12 months.

14. The method of claim 12 wherein the parameters K₁₁ to K₁₁ are selected from the following ranges:
K₁ is selected in a range from −1.04 to −0.41,
K₂ is selected in a range from −1.10 to −0.92,
K₃ is selected in a range from 0.89 to 1.26,
K₄ is selected in a range from 0.20 to 0.33,
K₅ is selected in a range from −0.03 to 0.03,
K₆ is selected in a range from 0.34 to 0.48,
K₇ is selected in a range from −0.94 to −0.76,
K₈ is selected in a range from 0.50 to 0.69,
K₉ is selected in a range from −0.08 to 0.13,
K₁₀ is selected in a range from 0.38 to 0.60,
K₁₁ is selected in a range from −0.25 to 0.15.

15. The method of claim 14, wherein the parameters K₁ to K₁₁ have the following values:
K₁=−0.7265,
K₂=−1.0102,
K₃=1.0748,
K₄=0.2639,
K₅=0.0011,
K₆=0.4108,
K₇=−0.8529,
K₈=0.5925,
K₉=0.0230,
K₁₀=0.4876,
K₁₁=−0.0508.

16. The method of claim 12, wherein analysis of the risk score includes a step of comparing said score with a threshold value, a score greater than the threshold value meaning than the recipient is considered as at risk of returning to dialysis.

17. The method of claims 14 and 16 taken in combination, wherein the threshold value is 4.04.

18. A product/computer program containing a set of instructions characteristic of implementation of the method of claim 1.

19. A processing system including a computation unit and an input interface, characterized in that said system includes means for implementing the method of claim 1.
